(19) United States
(12) Patent Application Publication
(10) Pub. No.: US 2010/0008880 A1
(43) Pub. Date: Jan. 14, 2010

(54) Clear Cosmetic Compositions And Methods Of Using

(76) Inventors: John Castro, Huntington Station, NY (US); Garrett Vanderover, Bellerose, NY (US); Milagro Serrano, Bayshore, NY (US); Tatyana Tabakman, Brooklyn, NY (US)

Correspondence Address: THE ESTEE LAUDER COS, INC, 155 PINELAWN ROAD, STE 345 S, MELVILLE, NY, 11747, US

(21) Appl. No.: 12/485,252

(22) Filed: Jun. 16, 2009

Related U.S. Application Data
(63) Continuation-in-part of application No. 11/737,441, filed on Apr. 19, 2007.
(60) Provisional application No. 60/746,270, filed on May 3, 2006.

(51) Int. Cl. A61K 8/72; A61Q 1/06

(52) U.S. Cl. 424/64

(57) ABSTRACT

The present invention provides a cosmetic composition containing a partially crystalline polyolefin homopolymer or copolymer wax and a solvent system. Specifically, the partially crystalline polyolefin homopolymer or copolymer wax is characterized by a refractive index ranging from about 1.4 to about 1.6, a viscosity measured at 170° C. ranging from about 5,000 mPas to about 15,000 mPas, a weight average molecular weight ranging from about 22,000 to about 28,000, and a polydispersity index ranging from about 2 to about 2.5. The solvent system is characterized by a refractive index that is matched with the refractive index of the wax and includes one or more solvents that are compatible with the wax for forming a solid, substantially clear, stick-shaped cosmetic product.

### CROSS-REFERENCE TO RELATED APPLICATIONS

This is a Continuation-in-Part of U.S. patent application Ser. No. 11/737,441 filed Apr. 19, 2007, which in turn claims priority to U.S. Provisional Patent Application No. 60/746,270 filed May 3, 2006.

### FIELD OF THE INVENTION

The present invention relates to skin care cosmetic compositions and methods. In particular, the present invention relates to novel cosmetic composition comprising clear polypropylene and/or polyethylene waxes and methods of use thereof for forming solid, substantially clear stick-shaped cosmetic products.

### BACKGROUND OF THE INVENTION

Traditional lipsticks often contain waxes, gellants, fillers, heavy oils and plasticizers for developing structure and creating a desired stiffness. Incorporation of waxes raises the melting point and therefore improves the physical stability of the composition. However, ingredients such as waxes that are used to provide the balance of rigidity and firmness to the structure have a disadvantage in that they contribute to opacity of the product. Therefore, there is a need to create clear lipsticks that have the desired balance of rigidity and firmness while remaining clear.

The art teaches various methods of creating stable, clear lipsticks. Polyamides have been used to help maintain clarity while achieving the desired stiffness in clear cosmetic compositions. For example, U.S. Pat. No. 3,148,125 discloses a clear lipstick in which the colorants are oil soluble or water soluble dyes, rather than pigments. The dyes are solubilized in a gel the base of which is polyamide resin. The water-soluble dyes are made compatible with the formulation by using lower aliphatic co-solvents.

However, polyamides exhibit various incompatibilities that make their use less than desirable. For example, polyamides are not compatible with a wide variety of oils, such as hydrocarbon oils, which are commonly used in cosmetic compositions. Moreover, polyamides are less tacky and therefore their adhesion properties are significantly impaired. As a result, polyamides are less desirable in products such as lipsticks which require minimally an initial phase of adhesion during the application of the product to the skin or hair. Furthermore, polyamides are difficult to use in compositions with high melting points such as lipsticks containing waxes.

Other substitutes for traditional waxes in lipsticks include dibutyl lauroyl glutamide (commercially available as G.P.-1), which is used to maintain clarity without compromising structure and stability. However, G.P.-1 used alone does not provide the desired amount of clarity without additional ingredients such as hydrocarbons, which at certain levels can affect the consistency of the composition by dissolving some of the existing wax. Gelling agents such as dibenzyl monosorbitol acetal, DMSA, have been used with lipophilic waxes to create substantially clear deodorant compositions. See U.S. Pat. No. 5,750,125.

Refractive index matching allows for better passage of light with less light scattering through compositions such as gel sticks. The resulting product therefore appears more clear or translucent as a packaged composition or when initially applied topically to the skin. However, such compositions are very difficult to formulate, especially for three component systems (active, solvent and gellant) in which refractive index matching is extremely difficult.

Therefore, there still remains a need for stable anhydrous lipstick compositions comprising waxes that do not destroy clarity.

### SUMMARY OF THE INVENTION

The present invention comprises a solid, substantially clear cosmetic composition comprising a partially crystalline polyolefin homopolymer or copolymer wax and a solvent system. The partially crystalline polyolefin homopolymer or copolymer wax is characterized by a refractive index ranging from about 1.4 to about 1.6, a viscosity measured at 170° C. of between about 5,000 and 15,000 mPa, a weight average molecular weight (Mw) ranging from about 22,000 to about 28,000, and a polydispersity index ranging from about 2 to about 2.5. The solvent system is characterized by a refractive index that is matched with the refractive index of the wax and comprises one or more solvents that are compatible with the wax, such as tridecyl trimellitate, triethylhexyl trimellitate, isododecane, isohexadecane, isoeicosane, polyisobutene, isodecyl neopentanoate, lauryl lactate, ethylhexyl palmitate, octyldodecyl behenate, isododecyl ethylhexanoate, polyglyceryl-2 diisostearate, synthetic jojoba oil, octyldodecyl myristate, isononyl isononanoate, isodecyl isononanoate, isotridecyl isononanoate, octyl stearate, dioctyl adipate, dioctyl adipate/octyl palmitate/octyl stearate, octyl stearate/octyl palmitate/dioctyl adipate, hexyl laurate, coco-caprylate/caprate, cetearyl isononanoate, isocetyl stearate, isopropyl myristate, isopropyl palmitate, propylene glycol dipelargonate, cetyl octanoate, tricaprylin, octyldodecyl myristate, isostearyl neopentanoate, decyl oleate, isodecyl oleate, octyldodecyl stearoyl stearate, caprylic/capric triglyceride, hydroxy-terminated polyurethane, natural squalane, isostearyl neopentanoate, octyldodecanol, oleyl lactate, oleyl alcohol, oleyl oleate, glycereth-7 triacetate, polyglyceryl-3 diisostearate, pentaerythrityl tetraisostearate, triglycerine diisostearate, diglycerol diisostearate/IPDI copolymer, ricinus communis (castor) seed oil, menthyl anthranilate, ethylhexyl salicylate, 2-ethylhexyl-4-methoxycinnamate and combinations thereof.

The above-described cosmetic composition can be readily formulated into a solid, substantially clear stick-shaped cosmetic product, by: (a) mixing the partially crystalline polyolefin homopolymer or copolymer wax within the solvent system to form a mixture; (b) pouring the mixture into a mold comprising one or more stick-shaped cavities; and (b) cooling said mixture to ambient temperature, thereby forming one or more solid, substantially clear stick-shaped cosmetic products.

### DETAILED DESCRIPTION

Except in operating and comparative examples, or where otherwise explicitly indicated, all numbers in this description indicating amounts or ratios of material or conditions of reaction, physical properties of materials and/or use are to be understood as modified by the word “about.” All amounts are by weight of the final composition, unless otherwise specified.

The present invention is directed to substantially clear solid cosmetic compositions. “Clear” it is meant in the usual dictionary definition of this term. Thus, a substantially clear stick as referred herein is sufficiently transparent to allow for ready viewing of objects behind it. By contrast, a translucent cosmetic stick, although allowing light to pass through, causes the light to be so scattered that it will be impossible to clearly identify objects behind the translucent stick. Opaque sticks do not permit the light to pass through. Thus, according to the present invention there is a distinction between, for example, “substantially clear” and “translucent” cosmetic compositions.

By stable, it is meant that the structure of the solid composition is physically stable to have the desired stiffness to form a solid and the composition is chemically stable such that the oily components incorporated within the composition do not leak or migrate out of the composition.

More specifically, in the present invention, it has been surprisingly found that combining a specific wax having a refractive index of between 1.4-1.6 with a solvent system such that the refractive index of the solvent system matches the refractive index of the wax results in a clear, stable composition suitable for forming solid, substantially clear stick-shaped cosmetic products, such as lipsticks, sunscreen sticks, etc.

### Wax

The wax of the present invention is a polyolefin wax modified with silane compounds and prepared using metallocene catalysts. Specifically, the polyolefin wax is a partially crystalline polyolefin homopolymer or copolymer wax modified free-radically with a silane compound and having a viscosity as measured at 170° C. of between 10 and 50,000 mPas and a heat of fusion>10 J/g, wherein the silane compound used for modification includes at least one olefin double bond and at least one alkoxy radical attached directly to silicon and wherein the polyolefin waxes used for modification have been prepared using a metallocene catalyst. The polyolefin wax of the present invention and the process of making such are provided in further detail in U.S. Patent Application No. 2005-0043455, which is incorporated by reference herein.

Preferably, the partially crystalline polyolefin homopolymer or copolymer wax of the present invention has a refractive index ranging from about 1.4 to about 1.6, a viscosity measured at 170° C. of between 10 and 50,000 mPas, a weight average molecular weight ranging from about 22,000 to about 28,000, and a polydispersity index ranging from about 2 to about 2.5. The refractive index of a material may be measured by using a refractometer and by employing conventional refractory techniques. A particularly preferred refractometer used in the present invention is Fisher Scientific's Abbe Refractometer Model No. 6182 (available from Fisher Scientific of Springfield, N.J.). The weight average molecular weight (Mw) of a polymeric material can be measured by using light scattering, small angle neutron scattering (SANS), X-ray scattering, and sedimentation velocity. The polydispersity index (PDI) is a measure of the distribution of individual molecular weights in a give sample of polymers, and is typically calculated as the ratio of weight average molecular weight (Mw) divided by the number average molecular weight (Mn).

The most preferred polyolefin wax of the present invention is a polypropylene wax that is commercially available under the trade name Licocene PP 1602 from Clariant Corporation at Charlotte, N.C., which has a viscosity measured at 170° C. of about 7,000 mPas, a weight average molecular weight of about 25,200 and a polydispersity index of about 2.25. The polyolefin wax is present in an amount of from 0.1% to 20%, preferably from 1% to 10% and most preferably from 2% to 5%.

### Solvent

The second essential element of the present invention is a solvent system. It has been surprisingly found that a solvent system that has a refractive index that matches the refractive index of the wax of the present invention surprisingly creates a substantially clear solid anhydrous composition while maintaining stability. However, the inventors of the present invention have discovered that simply matching the refractive index of the solvents with the wax, however, does not necessarily yield a clear, stable composition. For example, certain solvents are not compatible with the wax of the present invention and therefore may destroy the clarity and/or structural integrity of the resulting composition.

Solvents suitable for use in the present invention with the partially crystalline polyolefin homopolymer or copolymer wax include, but are not limited to: tridecyl trimellitate (e.g., Hatcol™ 5103 from Hatcol Chemical Corp. at Fords, N.J.), triethyhexyl trimellitate (e.g., Hatcol™ 200 from Hatcol Chemical Corp. at Fords, N.J.), isododecane (e.g., Permethyl™ 99A from Permethyl Corporation at Pottstown, Pa.), isohexadecane (e.g., Permethyl™ 10A from Permethyl Corporation at Pottstown, Pa.), isoeicosane (e.g., Permethyl™ 102A from Permethyl Corporation at Pottstown, Pa.), polyisobutene (e.g., Permethyl™ 104A, 106A, Presperse from Permethyl Corporation at Pottstown, Pa.), isodecyl neopentanoate (e.g., Dermol™ 105 from Alzo, Inc. at Matawan, N.J.), lauryl lactate (e.g., Dermol™ LL from Alzo, Inc. at Matawan, N.J.), ethylhexyl palmitate (e.g., Dermol™ 816 from Alzo, Inc. at Matawan, N.J.), octyldodecyl behenate (e.g., Dermol™ 2022 from Alzo, Inc. at Matawan, N.J.), isododecyl ethylhexanoate (e.g., Dermol™ 334 from Alzo, Inc. at Matawan, N.J.), polyglyceryl-2 diisostearate (e.g., Dermol™ DGDIS from Alzo, Inc. at Matawan, N.J.), synthetic jojoba oil (e.g., Wickenol™ 139 from Alzo, Inc. at Matawan, N.J.), octyldodecyl myristate (e.g., Wickenol™ 142 from Alzo, Inc. at Matawan, N.J.), isononyl isononanoate (e.g., Wickenol™ 151 from Alzo, Inc. at Matawan, N.J.), isodecyl isononanoate (e.g., Wickenol™ 152 from Alzo, Inc. at Matawan, N.J.), isotridecyl isononanoate (e.g., Wickenol™ 153 from Alzo, Inc. at Matawan, N.J.), octyl stearate (e.g., Wickenol™ 156 from Alzo, Inc. at Matawan, N.J.), dioctyl adipate (e.g., Wickenol™ 159 from Alzo, Inc. at Matawan, N.J.), dioctyl adipate/octyl palmitate/octyl stearate (e.g., Wickenol™ 161 from Alzo, Inc. at Matawan, N.J.), octyl stearate/octyl palmitate/dioctyl adipate (e.g., Wickenol™ 163 from Alzo, Inc. at Matawan, N.J.), hexyl laurate (e.g., Cetiol™ A from Cognis North America at Cincinnati, Ohio), coco-caprylate/caprate (e.g., Cetiol™ LC from Cognis North America at Cincinnati, Ohio), cetearyl isononanoate (e.g., Cetiol™ SN from Cognis North America at Cincinnati, Ohio), isocetyl stearate (e.g., Cetiol™ G-16S from Cognis North America at Cincinnati, Ohio), isopropyl myristate (e.g., Liponate™ IPM from Lipo Chemicals Inc. at Paterson, N.J.), isopropyl palmitate (e.g., Liponate™ IPP from Lipo Chemicals Inc. at Paterson, N.J.), propylene glycol dipelargonate, cetyl octanoate (e.g., Trivent™ OC-16 from Alzo, Inc. at Matawan, N.J.), tricaprylin (e.g., Trivent™ OC-G from Alzo, Inc. at Matawan, N.J.), octyldodecyl myristate (e.g., Bernel™ Ester NPDC from Bernel Chemical Co. at Tenafly, N.J.), isostearyl neopentanoate (e.g., Ceraphyl™ 375 from ISP Global Technologies Deutschland GmbH at Frechen, Germany or Dermol 185 from Alzo, Inc. at Matawan, N.J.), decyl oleate (e.g., Ceraphyl™ 140 from ISP Global Technologies Deutschland GmbH at Frechen, Germany), isodecyl oleate (e.g., Ceraphyl™ 140A from ISP Global Technologies Deutschland GmbH at Frechen, Germany), octyldodecyl stearoyl stearate (e.g., Ceraphyl™ 847 from ISP Global Technologies Deutschland GmbH at Frechen, Germany), caprylic/capric triglyceride (e.g., Lexol™ GT-865 from Inolex Chemicals at Philadelphia, Pa., Liponate GC-K from Lipo Chemicals Inc. at Paterson, N.J., or Myritol™ 318 from Cognis North America at Cincinnati, Ohio), hydroxy-terminated polyurethane (e.g., Polyolprepolymer-14 from Penederm Inc. at Foster City, Calif.), natural squalane, isostearyl neopentanoate (e.g., Bernel™ Ester 2014 from Bernel Chemical Co. at Tenafly, N.J.), octyldodecanol (e.g., Eutanol™ G from Cognis North America at Cincinnati, Ohio), oleyl lactate (e.g., Pelemol™ OL from Adinop Co., Ltd. at Bangkok, Thailand), oleyl alcohol (e.g., Novol™ from Croda Inc. at Edison, N.J.), oleyl oleate (e.g., Schercemol™ OLO from Noveon, Inc. at Cleveland, Ohio), glycereth-7 triacetate (e.g., Pelemol™ G7A from Adinop Co., Ltd. at Bangkok, Thailand), polyglyceryl-3 diisostearate (e.g., Synton™ PAO 100 from Uniroyal Chemical Co. at Middlebury, Conn.), pentaerythrityl tetraisostearate (e.g., Crodamol™ PTIS from Croda Inc. at Edison, N.J.), triglycerine diisostearate (e.g., Emerest™ 2452 from Cognis North America at Cincinnati, Ohio), oleyl lactate (e.g., Plurol™ Diisostearique from), diglycerol diisostearate/IPDI Copolymer (e.g., Polyderm™ PPI-DGDIS from Alzo, Inc. at Matawan, N.J.), ricinus communis (castor) seed oil, menthyl anthranilate (e.g., Neo Heliopan™ MA from Haarmann and Reimer at Teterboro, N.J.), ethylhexyl salicylate (e.g., Neo Heliopan™ OS from Haarmann and Reimer at Teterboro, N.J.), polyisobutylene (e.g., Indopol™ H-1900 from BP Amoco Chemical Co. at Texas City, Tex.), 2-ethylhexyl-4-methoxycinnamate (e.g., Parsol™ MCX from DSM Nutritional Products, Inc. at Parsippany, N.J. or Escalol™ 557 from International Specialty Products at Wayne, N.J.) and combinations thereof.

Preferably, the solvent system comprises one or more solvents selected from the group consisting of tridecyl trimellitate, triethyhexyl trimellitate, octyldodecanol, ethylhexyl palmitate, polyglyceryl-3 diisostearate, and combinations thereof. Most preferably, the solvent system of the present invention comprises tridecyl trimellitate. The solvents of the present invention may each be used alone or in any combination in the solvent system, as long as the total in the composition is between 1% to 90%, preferably from 10% to 80% and most preferably from 30% to 50% by total weight of the composition.

### Gellant

The present invention optionally comprises a gellant. The gellant of the present invention is selected from traditional gellants that do not destroy the refractive index of the composition of the present invention. Specifically, the gellant is selected from but not limited to dibutyl/laurol glutamide (e.g., GP-1 from Ajinomoto USA, Inc. at Fort Lee, N.J.), steroyl inulin (e.g., Rheopearl™ Series from Tomen America Inc. at New York, N.Y.), ethyl hexyl hydrostearate/triethylhexyl trimellitate/C30-45 olefin (e.g., Clearwax™ from Sensient (Cosmetic Technologies (LCW) at South Plainfield, N.J.), 12-hydroxystearic acid (commerce commercially available from Phoenix Chemical at Somerville, N.J), polyethylene at low concentrations below 1% (available from Alzo Int'l and Mineral and Pigment Solutions, Inc.), ethylenediamine/stearyl dimmer tallate copolymer (e.g., Uniclear™ 100VG available from Arizona Chemicals at Jacksonville, Fla.), polyamide resin (e.g., Macromelt™ 6212, Versamid™ 940, GAX™ 14-030, GAX™ 14-029, all available from Cognis North America at Cincinnati, Ohio), hydrogenated styrene/isoprene copolymer (e.g., Kraton™ G-1701 from Kraton Polymers LLC at Houston, Tex.), styrene-ethylene/butylene-styrene black copolymer (e.g., Kraton™ G-1650, Kraton™ G-1652, G-1702, all available from Kraton Polymers LLC at Houston, Tex.), and combinations thereof.

The gellant is used in an amount of from 0. 1% to 50%, preferably from 5% to 15%, and most preferably from 6% to 10% by weight of the composition.

### OTHER INGREDIENTS

Optional ingredients may be included within the composition of the present invention as long as the physical integrity of the anhydrous solid composition is not compromised and the refractive index of the composition remains between 1.4-1.6.

Pigments are selected from fluorescent, pearlescent and glitter materials. Although the refractive index may not be match the refractive index of the wax or solvent system of the present invention, the pigments of the present invention do not detract from the refractive index of the composition.

Pigments of the present invention are used in an amount of from 0.01 to 10%, preferably from 0.1% to 5%, and most preferably from 0.1% to 1%.

Preferred fragrances are the essential oils. Dermatological agents that may be used in our compositions include vitamins, anti-inflammatory agents, hydroxyacids, and the like, and mixtures thereof. Sunscreens that may be used include dioxybenzone, 2-ethylhexyl 2-cyano-3, 3-diphenylacrylate, 2-ethylhexyl p-methoxycinnamate, 2-ethylhyxyl salicylate, homosalate, menthyl anthranilate, oxybenzone, octyl dimethyl PABA, red petrolatum, titanium dioxide, ferulic acid esters, or mixtures thereof. Preferably the sunscreen is titanium dioxide, ferulic acid esters or mixtures thereof.

The solvent used in compositions of this invention may also comprise other ingredients that are commonly employed by one of skill in the art in compositions for application to the skin (e.g., stabilizers, emollients, microbial agents, antioxidants and the like).

Other biological active agents can be added to the present inventive compositions as desired, as long as the clarity and refractive index are not compromised. Routine experimentation can determine the amounts required to retain a stable composition. The type of biological active agent added, can be any which is beneficially used in a topical cosmetic or pharmaceutical composition. For example, the composition can further comprise moisturizing actives, antibacterials, anti-irritant agents, anti-inflammatory agents, antiaging agents, antiwrinkle agents, sunscreen agents, or vitamins.

The preferred composition is in the form of an anhydrous solid stick composition. This composition is made using a conventional lipstick mold known to those skilled in the art. Specifically, the lipstick is made by first mixing the partially crystalline polyolefin homopolymer or copolymer wax within the solvent system. Any conventional mixing methods may be employed. The composition is then allowed to cool to ambient temperature.

### METHOD OF USE

The present inventive compositions are particularly useful for formulating lipsticks, although it can also be used for formulating other stick-shaped cosmetic products, such as sunscreen sticks, foundation sticks, etc. The composition is administered or topically applied to the lips. It is suggested as an example that topical application range from about once per day to about twice daily. The amount of composition applied to the skin will vary depending upon coverage desired.

The following examples further illustrate the invention, but the invention is not limited thereto.

### EXAMPLES

The following compositions provide illustrative examples of preferred embodiments of the present invention, which contain various inventive compositions as described hereinabove in the form of anhydrous sticks. Additional gellants and emollients may be incorporated, as shown in the compositions in the following tables.

TABLE 1

| Trade Name | CTFA name | Wt % |
|---|---|---|
| Licocene PP 1602 | Polypropylene Wax | 5.0 |
| Liponate TDTM | Tridecyl Trimellitate | QS |
| Cab-O-Sil M-5 | Silica | 5.0 |
| Salacos 120H | Hydroxystearic Acid | 10.0 |
| Panalane L-14E | Hydrogenated Polyisobutene | 20.0 |
| Versagel ME 750 | Hydrogenated | 20.0 |
| | Polyisobutene//Ethylene/Propylene/Styrene | |
| | Copolymer//Butylenes/Ethylene/Styrene | |
| | Copolymer | |
| Eutanol G | Octyldodecanol | 10.0 |

TABLE 2

| Trade Name | CTFA name | Wt % |
|---|---|---|
| Licocene PP 1602 | Polypropylene Wax | 18.0 |
| Plurol Diisostearique | Polyglyceryl-3 Diisostearate | QS |
| Silicone 556 Fluid | Phenyl Trimethicone | 1.0 |
| Novol | Oleyl Alcohol | 1.0 |
| Ceraphyl 28 | Cetyl Lactate | 2.0 |
| Cosmol 43V | Polyglyceryl-2 Triisostearate | 20.0 |
| Ganex V-220 | PVP/Eicosene Copolymer | 1.0 |
| Cosmol 168 ARV | Dipentaerythrityl | 30.0 |
| | Hexahydroxystearate/Stearate/Rosinate | |

TABLE 3

| Trade Name | CTFA name | Wt % |
|---|---|---|
| Licocene PP 1602 | Polypropylene Wax | 20.0 |
| Dermol 816 | Octyl Palmitate | QS |
| Ceraphyl 28 | Cetyl Lactate | 2.0 |
| Bois II | Santalum Album (Sandalwood) Extract/ | 36.0 |
| | Phellodendron Amurense Bark Extract/ | |
| | Hordeum Distichon (Barley) Extract | |
| Ganex V-216 | PVP/Hexadecene Copolymer | 4.0 |
| Panalane H-300E | Hydrogenated Polyisobutene | 24.0 |

TABLE 4

| Trade Name | CTFA name | Wt % |
|---|---|---|
| Licocene PP 1602 | Polypropylene Wax | 20.0 |
| Eutanol G | Octyldodecanol | QS |
| Silicone 556 Fluid | Phenyl Trimethicone | 1.0 |
| Eutanol G-16 | Isocetyl Alcohol | 1.0 |
| Ceraphyl 28 | Cetyl Lactate | 2.0 |
| Bois II | Santalum Album (Sandalwood) Extract/ | 36.0 |
| | Phellodendron Amurense Bark Extract/ | |
| | Hordeum Distichon (Barley) Extract | |
| Ganex V-216 | PVP/Hexadecene Copolymer | 4.0 |
| Hest 2T-4HS | Ditrimethylolpropane | 24.0 |
| | Tetrahydroxystearate | |

Although the invention has been variously disclosed herein with reference to illustrative embodiments and features, it will be appreciated that the embodiments and features described hereinabove are not intended to limit the scope of the invention, and that other variations, modifications and other embodiments will suggest themselves to those of ordinary skill in the art. The invention therefore is to be broadly construed, consistent with the claims hereafter set forth.

What is claimed is:

1. A cosmetic composition comprising:
a partially crystalline polyolefin homopolymer or copolymer wax having a refractive index ranging from about 1.4 to about 1.6, a viscosity measured at 170° C. ranging from about 5,000 mPas to about 15,000 mPas, a weight average molecular weight ranging from about 22,000 to about 28,000, and a polydispersity index ranging from about 2 to about 2.5; and
a solvent system having a refractive index that is matched with the refractive index of the wax, wherein the solvent system comprises one or more solvents selected from the group consisting of tridecyl trimellitate, triethylhexyl trimellitate, isododecane, isohexadecane, isoeicosane, polyisobutene, isodecyl neopentanoate, lauryl lactate, ethylhexyl palmitate, octyldodecyl behenate, isododecyl ethylhexanoate, polyglyceryl-2 diisostearate, synthetic jojoba oil, octyldodecyl myristate, isononyl isononanoate, isodecyl isononanoate, isotridecyl isononanoate, octyl stearate, dioctyl adipate, dioctyl adipate/octyl palmitate/octyl stearate, octyl stearate/octyl palmitate/dioctyl adipate, hexyl laurate, coco-caprylate/caprate, cetearyl isononanoate, isocetyl stearate, isopropyl myristate, isopropyl palmitate, propylene glycol dipelargonate, cetyl octanoate, tricaprylin, octyldodecyl myristate, isostearyl neopentanoate, decyl oleate, isodecyl oleate, octyldodecyl stearoyl stearate, caprylic/capric triglyceride, hydroxy-terminated polyurethane, natural squalane, isostearyl neopentanoate, octyldodecanol, oleyl lactate, oleyl alcohol, oleyl oleate, glycereth-7 triacetate, polyglyceryl-3 diisostearate, pentaerythrityl tetraisostearate, triglycerine diisostearate, diglycerol diisostearate/IPDI copolymer, ricinus communis (castor) seed oil, menthyl anthranilate, ethylhexyl salicylate, 2-ethylhexyl-4-methoxycinnamate and combinations thereof.

2. The composition of claim 1, wherein the partially crystalline polyolefin homopolymer or copolymer wax comprises a polyethylene-propylene copolymer.

3. The composition of claim 1, wherein the solvent system comprises tridecyl trimellitate.

4. The composition of claim 1 further comprising a gellant selected from the group consisting of dibutyl/laurol glutamide, steroyl inulin, ethyl hexyl hydrostearate/triethylhexyl trimellitate/C30-45 olefin, 12-hydroxystearic acid, polyethylene, ethylenediamine/stearyl dimmer tallate copolymer, polyamide resin, hydrogenated styrene/isoprene copolymer, styrene-ethylene/butylene-styrene black copolymer, and combinations thereof.

5 . A solid, substantially clear stick-shaped cosmetic product comprising:
a partially crystalline polyolefin homopolymer or copolymer wax having a refractive index ranging from about 1.4 to about 1.6, a viscosity measured at 170° C. ranging from about 5,000 mPas to about 15,000 mPas, a weight average molecular weight ranging from about 22,000 to about 28,000, and a polydispersity index ranging from about 2 to about 2.5; and
a solvent system having a refractive index that is matched with the refractive index of the wax, wherein the solvent system comprises one or more solvents selected from the group consisting of tridecyl trimellitate, triethylhexyl trimellitate, isododecane, isohexadecane, isoeicosane, polyisobutene, isodecyl neopentanoate, lauryl lactate, ethylhexyl palmitate, octyldodecyl behenate, isododecyl ethylhexanoate, polyglyceryl-2 diisostearate, synthetic jojoba oil, octyldodecyl myristate, isononyl isononanoate, isodecyl isononanoate, isotridecyl isononanoate, octyl stearate, dioctyl adipate, dioctyl adipate/octyl palmitate/octyl stearate, octyl stearate/octyl palmitate/dioctyl adipate, hexyl laurate, coco-caprylate/caprate, cetearyl isononanoate, isocetyl stearate, isopropyl myristate, isopropyl palmitate, propylene glycol dipelargonate, cetyl octanoate, tricaprylin, octyldodecyl myristate, isostearyl neopentanoate, decyl oleate, isodecyl oleate, octyldodecyl stearoyl stearate, caprylic/capric triglyceride, hydroxy-terminated polyurethane, natural squalane, isostearyl neopentanoate, octyldodecanol, oleyl lactate, oleyl alcohol, oleyl oleate, glycereth-7 triacetate, polyglyceryl-3 diisostearate, pentaerythrityl tetraisostearate, triglycerine diisostearate, diglycerol diisostearate/IPDI copolymer, ricinus communis (castor) seed oil, menthyl anthranilate, ethylhexyl salicylate, 2-ethylhexyl-4-methoxycinnamate and combinations thereof

6. The cosmetic product of claim 5, wherein the partially crystalline polyolefin homopolymer or copolymer wax comprises a polyethylene-propylene copolymer.

7. The cosmetic product of claim 5, wherein the solvent system comprises tridecyl trimellitate.

8. The cosmetic product of claim 5, further comprising a gellant selected from the group consisting of dibutyl/laurol glutamide, steroyl inulin, ethyl hexyl hydrostearate/triethylhexyl trimellitate/C30-45 olefin, 12-hydroxystearic acid, polyethylene, ethylenediamine/stearyl dimmer tallate copolymer, polyamide resin, hydrogenated styrene/isoprene copolymer, styrene-ethylene/butylene-styrene black copolymer, and combinations thereof.

9. A method of making the solid, substantially clear stick-shaped cosmetic product of claim 5, comprising:
(a) mixing the partially crystalline polyolefin homopolymer or copolymer wax within the solvent system to form a mixture;
(b) pouring the mixture into a mold comprising one or more stick-shaped cavities; and
(b) cooling said mixture to ambient temperature, thereby forming one or more solid, substantially clear stick-shaped cosmetic products.

10. The method of claim 9, wherein the partially crystalline polyolefin homopolymer or copolymer wax is polyethylene-propylene copolymer.

11. The method of claim 9, wherein the solvent system comprises tridecyl trimellitate.

12. The method of claim 9, wherein the mixture further comprises a gellant selected from the group consisting of dibutyl/laurol glutamide, steroyl inulin, ethyl hexyl hydrostearate/triethylhexyl trimellitate/C30-45 olefin, 12-hydroxystearic acid, polyethylene, ethylenediamine/stearyl dimmer tallate copolymer, polyamide resin, hydrogenated styrene/isoprene copolymer, styrene-ethylene/butylene-styrene black copolymer, and combinations thereof.
